(19) United States
(12) Patent Application Publication
(10) Pub. No.: US 2026/0232850 A1
(43) Pub. Date: Aug. 13, 2026

(54) COMPOSITIONS AND METHODS FOR TARGETING TUMOR-ASSOCIATED MACROPHAGES

(71) Applicant: RESOLUTE SCIENCE, INC., San Diego (CA)

(72) Inventor: Faith Hemenway BARNETT, San Diego, CA (US)

(21) Appl. No.: 19/157,584

(22) PCT Filed: Feb. 21, 2024
(86) PCT No.: PCT/US2024/016752

§ 371 Date: Aug. 18, 2025

Related U.S. Application Data
(60) Provisional application No. 63/447,627, filed on Feb. 22, 2023.

(51) Int. Cl. A61K 51/04

(52) U.S. Cl. CPC A61K 51/0491; A61K 51/0497; A61K 2121/00

(57) ABSTRACT

The present disclosure relates to compositions that target tumor associated macrophages. The compositions disclosed here preferably comprise a glucan backbone, a tumor-associated macrophage targeting moiety, a targeting moiety linker, a payload and optionally a payload linker. The present disclosure also provides methods of making such compositions. The present disclosure also provides methods of treatment using such compositions.

### CROSS-REFERENCE TO RELATED APPLICATION

This application claims priority from U.S. provisional application No. 63/447,627, filed Feb. 22, 2023, entitled “COMPOSITIONS AND METHODS FOR TARGETING TUMOR-ASSOCIATED MACROPHAGES,” the contents of which are incorporated by reference in their entirety.

### BACKGROUND

Soft tissue sarcomas (STS) are rare but deadly cancers of children and adults. The American Cancer Society estimates about 13,000 new STS cases per year in the United States with about 5,130 expected deaths and a 5-year survival for metastatic disease of only 16%. Conventional treatments, including surgery, chemotherapy, and radiotherapy, have yielded limited treatment success for STS. For example, undifferentiated pleomorphic sarcoma (UPS), a highly aggressive adult sarcoma, has a median overall survival for metastatic UPS of only 15.5 months. An adolescent associated aggressive sarcoma, Ewing's sarcoma/primitive neuroectodermal tumor (PNET), has a five-year survival rate of 15% for metastatic cases. Treatment of solid tumors has recently improved with the advent of a broad array of targeted therapies, such as small molecules and biologics. Small molecules have excellent penetrance into solid tumors, yet many of them work by inhibiting signal transduction pathways leading to growth inhibition rather than direct tumor cytotoxicity. If the entire tumor is not killed, cancer cells can develop resistance to the small molecule. Biologics, such as antibody and antibody-drug conjugates (ADC) can have profound potency for certain malignancies and have favorable stability in blood. However, antibodies have limited penetrance into solid tumors, and tumor cells can develop resistance to ADCs through a variety of cellular modifications (Collins et al. “Acquired Resistance to Antibody-Drug Conjugates” Cancers (Basel), 2019, 11(3):394). Thus, new approaches are needed to overcome the limitations of the current state of the art and to further improve the treatment outcomes of STS. In addition, STS encompass over 50 different histologic and molecular subtypes, with each displaying variable clinical behavior (Katz et al., “More Than 50 Subtypes of Soft Tissue Sarcoma: Paving the Path for Histology-Driven Treatments” Am Soc Clin Oncol Educ Book, 2018, 38, 925-938). Due partly to this variability, current STS treatment options have yielded limited efficacy and there is no single or combination treatments that can consistently and effectively treat all STS subtypes.

CD206⁺ cells, particularly macrophages, have been targeted by various molecules in the hopes of delivering diagnostic and therapeutic to sites where such cells assemble. One example of such molecules is found in US 2017/0209584, entitled, “Compositions for Targeting Macrophages and Other CD206 High Expressing Cells and Methods of Treating and Diagnosis.” While the molecules disclosed in this reference and others may target the CD206⁺ cells of interest, the molecules suffer from a number of short comings.

### BRIEF SUMMARY

In one aspect, provided is a composition comprising i) a tumor-associated macrophage-targeting moiety; ii) a glucan backbone, wherein the glucan backbone comprises a plurality of backbone monomers; iii) an active component comprising a dodecane tetraacetic acid (DOTA) substrate, wherein the active component is coupled to the glucan backbone, and wherein the DOTA substrate is chelated to a radioisotope selected from the group consisting of: ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, and ³²P; and iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

In another aspect, provided is a method of treating a solid tumor comprising administering to a subject in need thereof a composition, wherein the composition comprises: i) a tumor-associated macrophage-targeting moiety; ii) a glucan backbone, wherein the glucan backbone comprises a plurality of backbone monomers; iii) an active component comprising a dodecane tetraacetic acid (DOTA) substrate, wherein the active component is coupled to the glucan backbone, and wherein the DOTA substrate is chelated to a radioisotope; and iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety. In some embodiments, the radioisotope is selected from the group consisting of: ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, and ¹⁷⁷Lu.

Also provided is a method of imaging tumors comprising administering to a subject in need thereof a composition, wherein the composition comprises: i) a tumor-associated macrophage-targeting moiety; ii) a glucan backbone, wherein the glucan backbone comprises a plurality of backbone monomers; iii) an active component comprising a dodecane tetraacetic acid (DOTA) substrate, wherein the active component is coupled to the glucan backbone, and wherein the DOTA substrate is chelated to a radioisotope; and iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

Also provided is a method of delivering an agent to a tumor-associated macrophage comprising contacting the macrophage with a composition described herein.

Also provided is a method of treating cancer, a granulomatous disease, or a nonmalignant tumor in a subject in need thereof, comprising administering to the subject a therapeutically effective amount of a composition described herein.

### BRIEF DESCRIPTION OF THE DRAWINGS

FIG. 1 is a graphic representation of NMR spectra of Compound A.

### DETAILED DESCRIPTION

The present disclosure relates to compositions that target tumor-associated macrophages and methods of using such compositions. The compositions disclosed herein comprise a glucan backbone, a tumor-associated macrophage-targeting moiety, a targeting moiety linker, an active component comprising a tetraacetic acid (DOTA) substrate chelated to a radioisotope, and optionally a payload linker. The present disclosure also provides methods of making and using such compositions.

### Chemical Definitions

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Carbamate” refers to the group —O—C(═O)—NH—. Unless specified otherwise, it is understood that the nitrogen atom of the carbamate group is unsubstituted (i.e., bears a hydrogen atom).

“Oxo” refers to the moiety ═O.

“Optionally substituted” unless otherwise specified means that a group may be unsubstituted or substituted by one or more (e.g., 1, 2, 3, 4, 5, 6, 7, 8, 9, 10, 11, or 12) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

### Compositions

The compositions disclosed here comprise various components, including a glucan backbone, a tumor-associated macrophage-targeting moiety, a targeting moiety linker, an active component comprising a dodecane tetraacetic acid (DOTA) substrate, wherein the active component is coupled to the glucan backbone and wherein the DOTA substrate is chelated to a radioisotope (e.g., ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, and ¹⁷⁷Lu), and optionally a payload linker. The arrangement of these components provides a composition or a pharmaceutically acceptable salt thereof that targets tumor-associated macrophages (TAMs). The disclosed compositions may use the TAMs as receiver cells to pick up, process, and deliver a radioligand, such as ¹⁷⁷Lu chelated to a DOTA substrate (¹⁷⁷Lu-DOTA), to the tumor environment. For example, sarcomas are characterized by abundant tumor-associated macrophages (TAMs) (Fujiwara et al., “Role of tumor-associated macrophages in sarcomas” Cancers (Basel), 2021, 13(5):1086). Anti-CD206 immunohistochemistry on human sarcoma tissue array, including 59 specimens encompassing 19 sarcoma subtypes, have been shown to have high and relatively uniform expression of this TAM receptor on essentially all specimens. The disclosed compositions may be internalized by cells (e.g., CD206⁺) present in tumor-associated macrophages. The ability to be internalized by cells present in tumor-associated macrophages allows for the disclosed compositions to deliver radioligands to disease sites where such cells assemble. The present application describes improved compositions and methods for treating cancers or other non-cancerous diseases, including delivering a radioligand (e.g., ¹⁷⁷Lu-DOTA) to a tumor-associated macrophage by contacting the tumor-associated macrophage with the disclosed composition. The compositions disclosed herein are larger than typical small molecules but smaller than antibody drug conjugates, allowing excellent penetration into targeted locations and minimal leakage to normal tissues, thereby limiting potential toxicities. It has been previously demonstrated that compositions comprising a DOTA moiety chelated to gadolinium can specifically deliver imaging agents to tumors in WO 2022/040580, the disclosure of which is incorporated herein by reference in its entirety. The compositions described herein, comprising radioligands such as ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, and ¹⁷⁷Lu chelated to a DOTA substrate, are designed to target tumor-associated macrophages and to deliver local radiation to the surrounding tumor in a seek and destroy approach. In addition, while rapidly dividing, mutating and heterogenous cancer cells often develop resistance to traditional therapies, terminally-differentiated macrophages are under no selective pressures to develop resistance to the treatment methods disclosed herein. With regard to safety and efficacy, radioisotopes, such as ⁶⁷Ga, ⁶⁸Ga, ²¹²Bi, 13I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, and ¹⁷⁷Lu, can be safely used in the treatment methods described herein. For example, ¹⁷⁷Lu has a 1.5 mm maximal range of activity and a suitable half-life of 6.7 days (Frost et al., “Comparative efficacy of 177Lu and 90Y for anti-CD20 pretargeted radioimmunotherapy in murine lymphoma xenograft models” PLoS One, 2015, 10(3):e0120561). ¹⁷⁷Lu emits both photons and beta particles. The photons allow for imaging of tumors by single photon computed tomography (SPECT) or SPECT-CT. The emitted beta particles deliver local radiation.

The disclosed composition may also target specific receptors, such as CD205 (DEC205), CD206, CD207 (langerin), CD209 (DC-SIGN), CD280 (ENDO180), and CD301 (MGL), which are present on a tumor or near a tumor, allowing targeted delivery of payloads to the tumor.

### Glucan Backbone

The compounds described here comprise a glucan backbone, which is a linear, branched, or circular oligosaccharide or polysaccharide comprising a plurality of glucose monomers. In some embodiments, the plurality of glucose monomers are linked predominantly by C-1→C-6 glycosidic bonds. Other linkages such as α-1,3 or α-1,4 linkages may also be present. In some embodiments, the plurality of glucose monomers are linked by α-1,6 and α-1,3 glycosidic bonds. In some embodiments, the plurality of glucose monomers are linked by a mixture of α-1,6 and α-1,4 glycosidic bonds. A glucan backbone may also be defined as a polymer of glucose wherein the position of glycosidic bonds is varied. A glucan backbone may comprise the alpha or the beta isomer of glucose or a mixture of alpha and beta isomers. Examples of glucan backbones include dextran, a linear or branched compound, and cyclodextrin, a circular glucan. It is to be understood that, in some embodiments, monomers labeled with, for example, a, b, c, or d, are interspersed within the construct. It is also to be understood that, in some embodiments, the constructs described herein can be block or interspersed, e.g., random.

A glucan backbone may vary in mass and molecular weight, as determined in part by the number of glucose monomers. In some embodiments, a glucan backbone may range in molecular weight from 1-30 kilodaltons (kDa). Preferred embodiments include glucan backbones of approximately 1 kDa, 3 kDa, 6 kDa, 10 kDa, 20 kDa, or 30 kDa. In some embodiments, the glucan backbone may range in molecular mass from 1,000 to 30,000 grams per mole (g/mol). In some embodiments, the glucan backbone may contain glucose monomers ranging from 5 to 167 in number. The glucan backbone can be linear, branched, circular, or combinations thereof. For example, dextran is an example of a linear or branched glucan backbone. Cyclodextrin is another example of a glucan backbone. The backbones described here can be substituted or unsubstituted. For example, a substituted cyclodextrin is a cyclodextrin derivative that is hydrophobic, hydrophilic, ionized, non-ionized, or any other variation thereof.

In some embodiments, the glucan backbone comprises a plurality of backbone monomers, wherein the plurality of backbone monomers comprises a plurality of D-glucose monomers in a α-1,6 glycosidic linkage or beta-1,4 glycosidic linkage. In some embodiments, the plurality of D-glucose monomers is n, wherein n=16 to 111. In some embodiments, n=50 to 65. In some embodiments, the glucan backbone is a linear dextran molecule. In some embodiments, the glucan backbone is a cyclodextrin molecule, comprising 6 to 16 D-glucose monomers.

### Targeting Moiety

The compositions disclosed herein comprise a targeting moiety (e.g., a tumor-associated macrophage-targeting moiety) coupled to a glucan backbone. In some embodiments, the targeting moiety binds to a receptor, including but not limited to, CD205 (DEC205), CD206, CD207 (langerin), CD209 (DC-SIGN), CD280 (ENDO180), or CD301 (MGL). In some embodiments, the targeting moiety is a CD205 targeting moiety. In some embodiments, the targeting moiety is a CD206 targeting moiety. In some embodiments, the targeting moiety is a CD207 targeting moiety. In some embodiments, the targeting moiety is a CD209 targeting moiety. In some embodiments, the targeting moiety is a CD280 targeting moiety. In some embodiments, the targeting moiety is a CD301 targeting moiety. In some embodiments, the target receptor is on a tumor-associated macrophage. In some embodiments, the target receptor is on a cancer or tumor cell. In some embodiments, the targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate. In some embodiments, the targeting moiety comprises a mannose, including D- and L-isomers thereof. In some embodiments, the targeting moiety comprises a furanose. In some embodiments, the targeting moiety comprises a pyranose. In some embodiments, the targeting moiety is D-mannose. It is understood that the tumor-associated macrophage-targeting moiety may be referred to as the targeting moiety.

In some embodiments, the targeting moieties are attached to between about 10% and about 50% of the glucose residues of the glucan backbone, or between about 20% and about 45% of the glucose residues, or between about 25% and about 40% of the glucose residues. (It should be noted that with reference to a collection or population of the compositions described herein, the MWs referenced herein, as well as the number and degree of conjugation of receptor substrates, leashes, and therapeutic moieties attached to the dextran backbone refer to average amounts for a given quantity of carrier molecules, since the synthesis techniques will result in some variability.)

### Ratio of Targeting Linker to Backbone

The density of a targeting moiety relative to backbone subunits is presented using a targeting moiety to backbone subunit ratio for linear, branched, or circular polysaccharide backbones. For example, degree of substitution (d.s.) is used to communicate the density of targeting moieties on a glucan backbone. The ratio of a targeting moiety to a glucan backbone refers to the number of targeting moieties that substitute a backbone subunit or subunits. For example, a ratio of 1:7 or 1 to 7 means that there is one targeting moiety for every seven glucose subunits in a glucan backbone. The d.s. describes the average number of substituents or substituted positions per unit base. For example, a d.s. of 0.9 means that one backbone subunit is substituted with an average of 0.9 targeting moieties. In some embodiments, the targeting moiety to backbone subunit ratio is from about 1:5 to about 1:25. In some embodiments, the targeting moiety to backbone subunit ratio is from at least 1 to 50 (e.g., at least 1 to 33, at least 1 to 35, at least 1 to 40, or at least 1 to 45) to about 1:5. In some embodiments, the targeting moiety to backbone subunit ratio is from about 1:16 to about 1:167. In some embodiments, the targeting moiety to backbone subunit ratio is about 1:33. In some embodiments, the targeting moiety to backbone subunit ratio is from about 1:6 to about 1:19. In some embodiments, the d.s. is from about 0.1 to about 7. In some embodiments, the d.s. is from about 0.5 to 5. In some embodiments, in conjunction with the embodiments above or below, the targeting moiety comprises a mannose.

### Targeting Linker

A targeting linker is a cleavable or a non-cleavable linker that connects a glucan backbone to a targeting moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The targeting linker may comprise a carbamate group. In some embodiments, the targeting linker comprises a carbamate group and a chain moiety, wherein the carbamate group is connected to a backbone monomer and the chain moiety connects the carbamate group and the targeting moiety. In some embodiments, the chain moiety of the targeting linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more targeting moieties are attached to the glucan backbone through a linker. The linker may be attached at from about 1 to about 50% of the backbone moieties. In some embodiments, the targeting linker comprises a C₁₋₁₂ alkylene chain and a carbamate group, wherein the carbamate group is connected to the backbone monomer and the C₁₋₁₂ alkylene chain connects the carbamate group and the targeting moiety.

### Active Component

An active component is a molecule or a compound that may be used for therapeutic purposes. An active component is also referred to as a payload. In some embodiments, the active component comprises a dodecane tetraacetic acid (DOTA) substrate, wherein the active component is coupled to the glucan backbone and wherein the DOTA substrate is chelated to a radioisotope. In some embodiments, the radioisotope emits photons. In some embodiments, the radioisotope emits beta particles. In some embodiments, the radioisotope emits both photons and beta particles. In some embodiments, the radioisotope is ⁶⁷Ga. In some embodiments, the radioisotope is ⁶⁸Ga. In some embodiments, the radioisotope is ²¹²Bi. In some embodiments, the radioisotope is ¹³¹I. In some embodiments, the radioisotope is ¹¹¹In. In some embodiments, the radioisotope is ⁹⁰Y. In some embodiments, the radioisotope is ¹⁸⁶Re. In some embodiments, the radioisotope is ²¹¹At. In some embodiments, the radioisotope is ¹²⁵I. In some embodiments, the radioisotope is ¹⁸⁸Re. In some embodiments, the radioisotope is ¹⁵³Sm. In some embodiments, the radioisotope is ²¹³Bi. In some embodiments, the radioisotope is ³²P. In some embodiments, the radioisotope is ²²⁵Ac. In some embodiments, the radioisotope is ¹⁷⁷Lu. In some embodiment, the DOTA substrate is optionally substituted. In some embodiments, the DOTA substrate is a DOTA analogue.

In some embodiments, the active component is a radioligand, wherein the radioligand comprises a dodecane tetraacetic acid (DOTA) substrate and a radioisotope (e.g., ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, or ¹⁷⁷Lu), wherein the radioisotope is chelated to the DOTA substrate. In some embodiments, the radioligand may be used for imaging of tumors, delivering local radiation, or a combination thereof. For example, ¹⁷⁷Lu emits both photons and beta particles. The photons allow for imaging of tumors by single photon computed tomography (SPECT). The emitted beta particles deliver local radiation.

In some embodiments, the active component comprises a radioligand of Formula (A), wherein X of Formula (A) is a radio isotope selected from the group consisting of ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, and ¹⁷⁷Lu, wherein Formula (A) is:

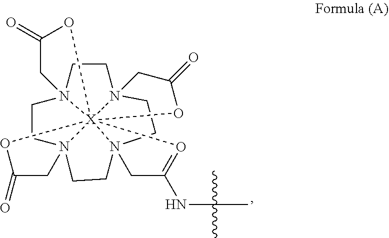

or an ionized form or pharmaceutically acceptable salt thereof,
In some embodiments, the active component comprises a radioligand of Formula (B):

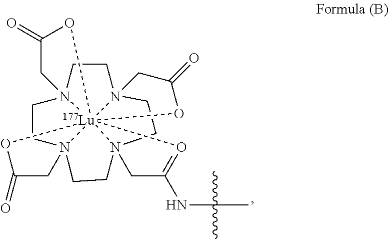

or an ionized form or pharmaceutically acceptable salt thereof.

### Payload Linker

In certain embodiments the active component or payload is coupled directly to the glucan backbone. In some embodiments, the active component is connected to a glucan backbone via a linker. The linker can be cleavable or non-cleavable. In some embodiments, the one or more therapeutic agent is attached via a biodegradable linker. In some embodiments, the biodegradable linker is acid sensitive, such as a hydrazone linker. The use of an acid sensitive linker enables the drug to be transported into the cell and allows for the release of the drug substantially inside of the cell. In some embodiments, the payload linker is a Val-Cit linker.

The payload linker may comprise a carbamate group. In some embodiments, the payload linker comprises a carbamate group and a chain moiety, wherein the carbamate group is connected to a backbone monomer and the chain moiety connects the carbamate group and the active component. In some embodiments, the chain moiety of the payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the molar ratio between the targeting moiety (e.g., mannose) and the active component (e.g., radioligand) is from about 1:10 to about 10:1. In some embodiments, the molar ratio between the targeting moiety and the active component is at least about 1:10 (e.g., about 1:9, 1:8, 1:7, 1:6, 1:5, 1:4, 1:3, 1:2, 1:1, 2:1, 3:1, and 4:1). In some embodiment, the molar ratio between the targeting moiety and the active component is about 1:1. In some embodiment, the molar ratio between the targeting moiety and the active component is about 1:2. In some embodiment, the molar ratio between the targeting moiety and the active component is about 1:3. In some embodiment, the molar ratio between the targeting moiety and the active component is about 1:4.

In some embodiments, the payload linker comprises a —C(O)—C₁₋₁₂ alkylene chain and a carbamate group, wherein the carbamate group is connected to the backbone monomer and the —C(O)—C₁₋₁₂ alkylene chain connects the carbamate group and the payload.

### Secondary Payloads and Linkers

In addition to the payloads, the compounds disclosed here can encompass the inclusion of secondary agents that can be coupled to the glucan backbone to add additional functional capabilities. Typically, the secondary payload is coupled to the linker in any manner similar to what may be used to couple the targeting moiety to the targeting linker.

In some embodiments, a secondary payload may be a radiosensitizer. See, e.g., Zhang et al., “Application of Radiosensitizers in Cancer Radiotherapy” Int J Nanomedicine, 2021, 16:1083-1102. In some embodiments, the radiosensitizer is a small molecule radiosensitizer including, but not limited to, monomethyl auristatin E (MMAE), mitomycin C, misonidazole, curcumin, or paclitaxel. In some embodiments, a secondary payload may be an anti-tuberculosis medicine (e.g., rifampin or isoniazid).

A secondary payload can encompass, for example, additional agents for imaging, therapy, or for other purposes. Specifically, in one embodiment, combinations of therapeutic and imaging agents can be linked to the glucan backbone to combine diagnostic and therapeutic functionalities. In another embodiment, various amino acids, such as cysteine or lysine can be coupled to the linker to crosslink the molecule to a target.

A secondary payload linker is a cleavable or a non-cleavable linker that connects a glucan backbone to a secondary payload moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The secondary payload linker may comprise a carbamate group. In some embodiments, the secondary payload linker comprises a carbamate group and a chain moiety, wherein the carbamate group is connected to a backbone monomer and the chain moiety connects the carbamate group and the secondary agent. Herein, a carbamate functional group takes the plain and ordinary meaning derived from the field of organic chemistry. In some embodiments, the chain moiety of the secondary payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more secondary payload moieties are attached to the glucan backbone through a linker. The linker may be attached at from about 1 to about 50% of the backbone moieties.

In some embodiments, the composition described herein comprises a compound of Formula (I):

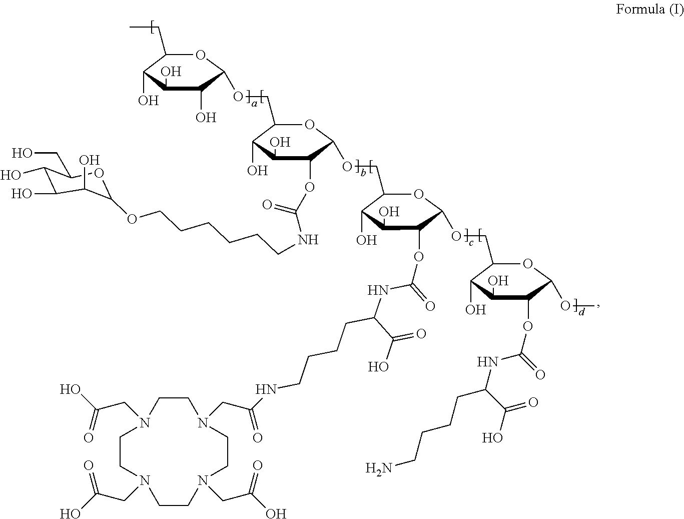

or a pharmaceutically acceptable salt thereof, wherein the DOTA substrate of Formula (I) is chelated to a radioisotope. In some embodiments, the radioisotope is ⁶⁷GA. In some embodiments, the radioisotope is ⁶⁸GA. In some embodiments, the radioisotope is ²¹²Bi. In some embodiments, the radioisotope is ¹³¹I. In some embodiments, the radioisotope is ¹¹¹In. In some embodiments, the radioisotope is ⁹⁰Y. In some embodiments, the radioisotope is ¹⁸⁶Re. In some embodiments, the radioisotope is ²¹¹At. In some embodiments, the radioisotope is ¹²⁵I. In some embodiments, the radioisotope is ¹⁸⁸Re. In some embodiments, the radioisotope is ¹⁵³Sm. In some embodiments, the radioisotope is ²¹³Bi. In some embodiments, the radioisotope is ³²P. In some embodiments, the radioisotope is ²²⁵Ac. In some embodiments, the radioisotope is ¹⁷⁷Lu. In some embodiments, a, b, c, and d of Formula (I) may each independently refer to an integer of 0, at least 1, from about 1 to about 165, from about 16 to about 111, from about 50 to about 65, or from about 6 to about 6. It is to be understood that monomers of the types labelled with a, b, c, or d may be in a block co-polymer arrangement or may be interspersed (e.g., randomly arranged) within the polymer or any combination thereof unless otherwise indicated. Groups not specified in the above schemes, such as any end groups to the glucan backbone, may be any end groups recognizable by one skilled in the art. It is to be understood that the glucan backbone of Formula (I) may be linear, branched, or circular. In some embodiments, an end group of the glucan backbone of Formula (I) may be an hydroxy end group of the monomer. A compound comprising ¹⁷⁷Lu chelated to the compound of Formula (I) is referred to as Compound A:

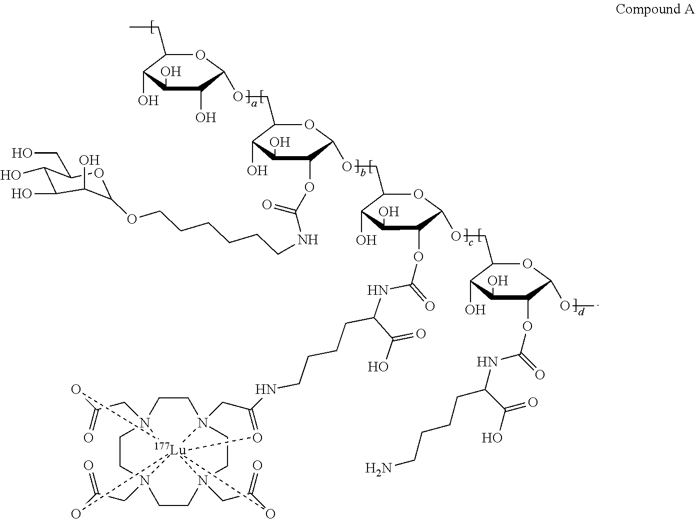

In some embodiments, monomers of the types labelled with a, b, c, or d in the compound of Formula (I) may be in a block co-polymer arrangement or may be interspersed (e.g., randomly arranged) within the polymer or any combination thereof unless otherwise indicated. In some embodiments, a, b, c, and d of Formula (I) may each independently refer to an integer of 0, at least 1, from about 1 to about 165, from about 16 to about 111, from about 50 to about 65, or from about 6 to about 6. In some embodiments, the glucan backbone of Formula (I) is linear, branched, circular, or combinations thereof. In some embodiments, an end group of the glucan backbone of Formula (I) may be a hydroxy end group of the monomer. In some embodiments, an end group of the glucan backbone of Formula (I) may be any end groups recognizable by one skilled in the art.

In some embodiments, the glucan backbone of Formula (I) is linear, branched, circular, or combinations thereof; a, b, c, and d is each independently elected from the group consisting of: an integer of 0, an integer of at least 1, an integer from about 1 to about 165, an integer from about 16 to about 111, an integer from about 50 to about 65, and an integer from about 6 to about 16; the end group of the glucan backbone is a hydroxy end group of the monomer; and the radioisotope is ¹⁷⁷Lu. In some embodiments, the glucan backbone of Formula (I) is circular; a, b, c, and d is each independently elected from the group consisting of: an integer of 0, an integer of at least 1, an integer from about 1 to about 165, an integer from about 16 to about 111, an integer from about 50 to about 65, and an integer from about 6 to about 16; and the radioisotope is ¹⁷⁷Lu. In some embodiments, the glucan backbone of Formula (I) is linear; a, b, c, and d is each independently elected from the group consisting of: an integer of 0, an integer of at least 1, an integer from about 1 to about 165, an integer from about 16 to about 111, an integer from about 50 to about 65, and an integer from about 6 to about 16; the end group of the glucan backbone is a hydroxy end group of the monomer; and the radioisotope is ¹⁷⁷Lu. In some embodiments, the glucan backbone of Formula (I) is branched; a, b, c, and d is each independently elected from the group consisting of: an integer of 0, an integer of at least 1, an integer from about 1 to about 165, an integer from about 16 to about 111, an integer from about 50 to about 65, and an integer from about 6 to about 16; the end group of the glucan backbone is a hydroxy end group of the monomer; and the radioisotope is ¹⁷⁷Lu.

In some embodiments, in combination with the embodiments above or below, the glucan backbone of Formula (I) is about 6 kDa, wherein the glucan backbone is a dextran. In some embodiments, in combination with the embodiments above or below, the a, b, and c groups of Formula (I) are interspersed. In some embodiments, in combination with the embodiments above or below, the end groups of the glucan backbone is a natural end group of glucose, such as a hydroxy end group. In some embodiments, in combination with the embodiments above or below, the ratio of the targeting moiety to backbone monomers of Formula (I) is about 1:30 to 1:40 (e.g., 1:33) or about 1:33 to 1:40 (e.g., 1:37). In some embodiments, in combination with the embodiments above or below, the ratio of ¹⁷⁷Lu-DOTA to mannose of Formula (I) is about 1:1 to 1:3 (e.g., 1:1) or about 1:3 to 1:5 (e.g., 1:4).

In some embodiments, provided herein is a compound of Formula (I) wherein the glucan backbone is a dextran; the molecular weight of the glucan backbone is about 6 kDa; the a, b, and c groups are interspersed; the end groups of the glucan backbone is a natural end group of glucose, such as a hydroxy end group; the ratio of the targeting moiety to backbone monomers of is about 1:30 to 1:40 (e.g., 1:33); and the ratio of ¹⁷⁷Lu-DOTA to mannose is about 1:1 to 1:3 (e.g., 1:1).

In some embodiments, provided herein is a compound of Formula (I) wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:33 to 1:40 (e.g., 1:37); and the ratio of ¹⁷⁷Lu-DOTA to mannose is about 1:3 to 1:5 (e.g., 1:4).

### Therapeutic Methods

The compositions disclosed herein may be used to treat cancer or non-malignant tumors (e.g., meningiomas hemangioblastomas or giant cell tumors) in a subject in need thereof. Treatment comprises administering to the subject a therapeutically effective amount of a composition disclosed herein or a pharmaceutically acceptable salt thereof. The compositions disclosed herein may be used to treat a granulomatous disease (e.g., tuberculosis). The compositions disclosed herein may also be used to treat a tumor, such as a solid tumor or a benign tumor. In some embodiments, the compositions described herein may be used for imaging, treatment, or combinations thereof. For example, Lutetium-177 emits both photons, which allow for imaging of tumors by single photon computed tomography (SPECT), and beta particles, which deliver local radiation. In some embodiments, a composition comprising ¹⁷⁷Lu may be used for imaging of tumors by single photon computed tomography (SPECT), delivering local radiation, or combinations thereof. In some embodiments, a composition comprising ⁶⁷Ga may be used for imaging of tumors by single photon computed tomography (SPECT), delivering local radiation, or combinations thereof. In some embodiments, a composition comprising ⁶⁸Ga may be used for imaging of tumors by single photon computed tomography (SPECT), delivering local radiation, or combinations thereof.

In some embodiments, the tumor is a solid tumor. In some embodiments, the solid tumor is sarcoma, lymphoma, carcinoma, blastoma, germ cell tumor, B cell lymphoma, T cell lymphoma, mycosis fungoides, Hodgkin's Disease, myeloid leukemia, multiple myeloma, bladder cancer, brain cancer, nervous system cancer, head and neck cancer, squamous cell carcinoma of head and neck, kidney cancer, lung cancers, small cell lung cancer, non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, triple negative breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, gliosarcoma, Kaposi sarcoma, esophageal cancer, hepatocellular cancer, pancreatic cancer, Merkel cell carcinoma, multiple myeloma, acute myeloid leukemia (AML), hemangioblastomas, and schwannomas. In some embodiments, the solid tumor is sarcoma or glioblastoma. In some embodiments, the solid tumor is soft tissue sarcomas.

In some embodiments, the tumor is a benign tumor, such as meningioma. In some embodiments the meningioma is a grade 1, grade 2, or grade 3 meningioma.

Methods of treating or preventing diseases or disorders are provided using the disclosed compositions. The disclosed compositions can be used for targeting CD205 (DEC205), CD206, CD207 (langerin), CD209 (DC-SIGN), CD280 (ENDO180), or CD301 (MGL), which are present on a tumor or near a tumor. The disclosed compounds can be used for targeting of macrophages for treatment of intracellular pathogens (M. tuberculosis, F. tularensis, S. typhi). The disclosed compounds can be used to target tumor-associated macrophages, e.g. to be used for treating cancer. Macrophage-related and other CD205 (DEC205), CD206, CD207 (langerin), CD209 (DC-SIGN), CD280 (ENDO180), or CD301 (MGL) high expressing cell-related diseases for which the compositions and methods herein may be used include, but are not limited to: acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, histocytosis, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with Streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, Rosai-Dorfman disease, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Also provided are methods of treatment comprising the administration of an effective amount of the compound and/or pharmaceutical composition for the treatment of a lysosomal storage disease. For example, a lysosomal storage disease includes, but is not limited to, Cholesterly ester storage disease, Wolman disease, Hunter syndrome, Hurler's disease, Fabry disease, Gaucher disease, Krabbe disease (globoid cell leukodystrophy) Metachromatic leukodystrophy, Niemann-Pick disease, Sandhoff disease, Tay-Sachs disease, Batten disease, Cystinosis, Danon disease, and Pompe disease.

One of ordinary skill in the art will appreciate that various kinds of molecules and compounds be delivered to a cell or tissue using the disclosed compounds.

In one aspect, provided herein is a method of treating tuberculosis comprising administering to a subject in need thereof a compound as described herein.

In another aspect, in combination with the aspects above or below, provided here is a method of imaging tumors, comprising administering to a subject in need there of a composition described herein.

In another aspect, provided herein is a method of treating a macrophage-mediated disorder comprising administering to a subject in need thereof an effective amount of a compound as described herein; and detecting the detection label at a predetermined location in the subject.

In another aspect, provided herein is a method of treating a macrophage-mediated disorder comprising administering to a subject in need thereof an effective amount of a compound as described herein.

In another aspect, provided herein is a method of targeting tumor-associated macrophages comprising administering to a subject in need thereof an effective amount of a compound as described herein.

In another aspect, provided herein is a method according to any of those described herein, wherein a linker is used to attach the one or more targeting moieties and one or more therapeutic agents.

In another aspect, provided herein is a method according to any of those described herein, wherein the disorder is cancer.

In another aspect, provided herein is a method according to any of those described herein, wherein the cancer is a sarcoma, lymphoma, leukemia, carcinoma, blastoma, melanoma, or germ cell tumor.

### Combination Therapy

In some embodiments, the compositions disclosed herein may be administered in combination with a second therapeutic agent. In some embodiments, the compositions disclosed herein is co-administered to a patient with a second therapeutic agent, an adjuvant therapy, or radiation therapy. In some embodiments, at least two compounds or compositions are administered to the patient at the same time, such that effective amounts or concentrations of each of the two or more compounds may be found in the patient at a given point in time. Although compositions according to the present disclosure may be co-administered to a patient at the same time, co-administration embraces both administration of two or more agents at the same time or at different times, provided that effective concentrations of all co-administered compounds or compositions are found in the subject at a given time. In certain preferred aspects of the present disclosure, one or more of the present compositions described above, are co-administered in combination with at least one additional bioactive agent.

### Administration

The compositions as described herein may in certain embodiments be administered in single or divided unit doses by the oral, parenteral or topical routes. Administration of the compositions may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, by inhalation spray, rectally, vaginally, or via an implanted reservoir, among other routes of administration. The most effective dosage form will depend upon the pharmacokinetics of the particular agent chosen as well as the severity of disease in the patient.

The disclosed compositions can be administered via any suitable method. The disclosed compositions can be administered parenterally into the parenchyma or into the circulation so that the disclosed compounds reach target tissues (e.g., where cancer cells may be located). The disclosed compositions can be administered directly into or adjacent to a tumor mass. The disclosed compositions can be administered intravenously. In still other embodiments, the disclosed compositions can be administered orally, intraperitoneally, intramuscularly, subcutaneously, intracavity, or transdermally.

Parenteral administration of the compounds, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

### Formulation

In an additional aspect, the description provides therapeutic or pharmaceutical compositions comprising an effective amount of a composition according to the present disclosure, optionally in combination with a pharmaceutically acceptable carrier, additive or excipient.

A typical formulation is prepared by mixing the compositions of the present disclosure with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the compound is being applied. Other pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives.

A patient or subject in need of therapy using compositions according to the present disclosure can be treated by administering to the patient (subject) an effective amount of the composition according to the present disclosure. The composition is included in the pharmaceutically acceptable carrier or diluent in an amount sufficient to deliver to a patient a therapeutically effective amount for the desired indication, without causing serious toxic effects in the patient treated. It should be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

### Kits

In some aspects, kits for use in cancer treatment are provided. Such kits can include a composition described herein.

In some embodiments, the kit can include instructions for use in any of the therapeutic methods described herein. The included instructions can provide a description of administration of the pharmaceutical compositions to a subject to achieve the intended activity, e.g., treatment of a disease or condition such as cancer, in a subject. In some embodiments, the instructions relating to the use of the pharmaceutical compositions described herein can include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

In some embodiments, the kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. In some embodiments, a kit can have a sterile access port (for example, the container can be an intravenous solution bag or a vial having a stopper pierceable by a hypodermic injection needle).

In some embodiments, the kits provided herein include an additional therapeutic agent useful in treating a disease as described herein.

### General Synthetic Methods

Compositions of the present disclosure will now be described by reference to illustrative synthetic schemes for their general preparation below and the specific examples that follow. Artisans will recognize that, to obtain the various compositions herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate.

Chromatography, recrystallization and other conventional separation procedures may also be used with intermediates or final products where it is desired to obtain a particular isomer of a compound or to otherwise purify a product of a reaction.

General methods of preparing compositions described herein are depicted in exemplified methods below.

In some embodiments, the compositions of described herein can be synthesized according to the procedure as shown in Scheme A1.

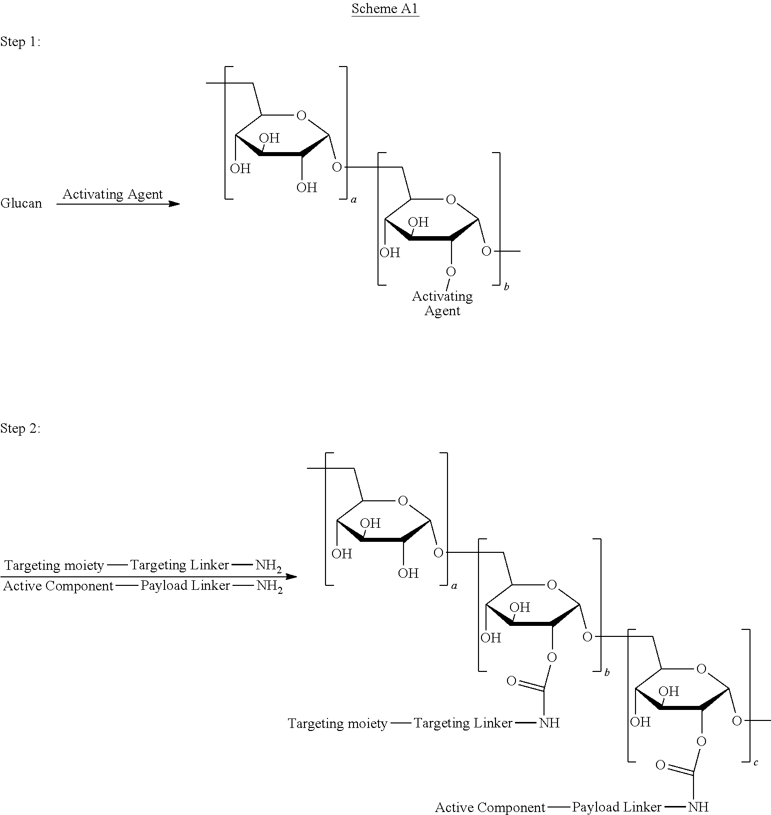

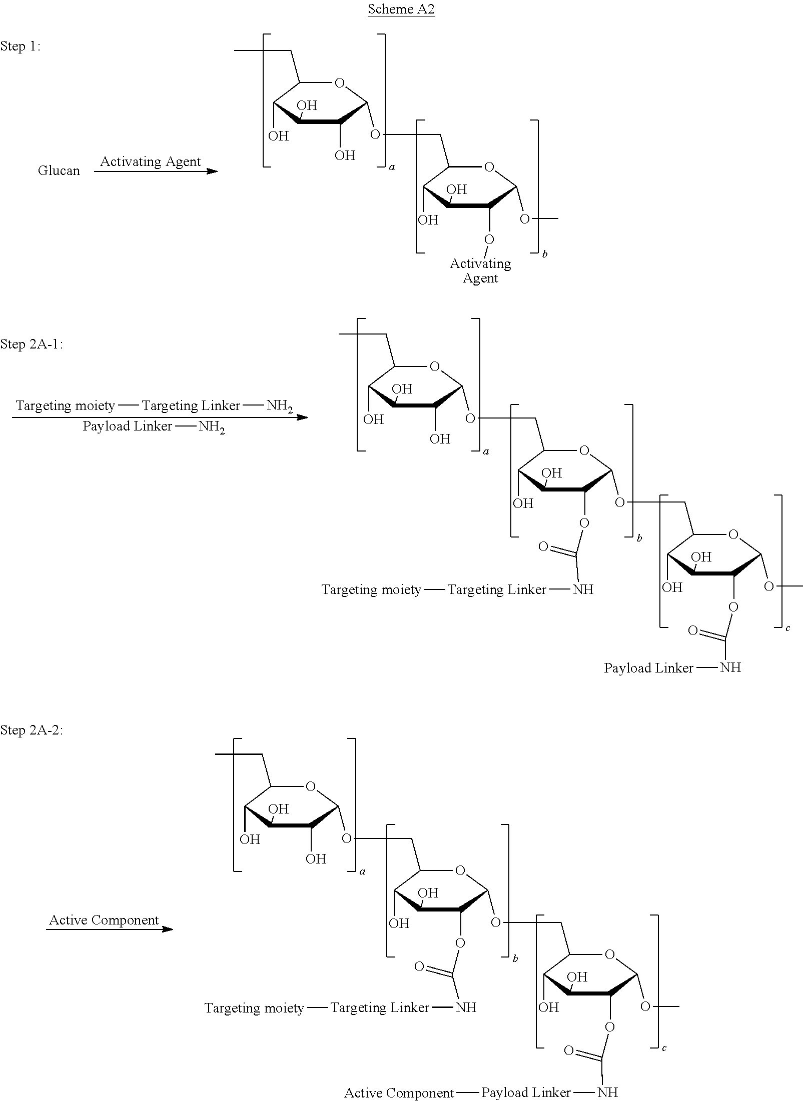

As can be seen in the above schemes, a glucan compound (such as a dextran or a cyclodextrin) is reacted with an activating agent. The resulting activated glucan derivative can then be reacted with the appropriate reagents to introduce a targeting moiety coupled to the glucan backbone via a targeting linker, as well as an active component linked to the glucan backbone via a payload linker. A skilled artisan will recognize that the above schemes are illustrative and that the various reagents and order of synthetic steps can be varied as required for obtaining the intended final products. For example, a, b, c, and d may each independently refer to an integer of 0, at least 1, at least 1, from about 1 to about 165, from about 16 to about 111, from about 50 to about 65, or from about 6 to about 16. It is to be understood that monomers of the types labelled with a, b, c, or d may be in a block co-polymer arrangement or may be interspersed (e.g., randomly arranged) within the polymer or any combination thereof unless otherwise indicated. It is also to be understood that the glucan backbone in the above scheme may be linear, branched, circular, or combinations thereof. Groups not specified in the above schemes, such as any end groups to the glucan backbone, may be any end groups recognizable by one skilled in the art. For example, an end group of the glucan backbone may be a hydroxy end group of the monomer.

### EXAMPLES

The following examples are included for illustrative purposes only and are not intended to limit the scope of the disclosure.

### Example 1. Synthesis of Compound a, a Targeted Single Photon Imaging and Radiotherapeutic Agent Comprising DOTA and Lutetium-177

A ¹⁷⁷Lu-labeled construct comprised of a targeting element, a dextran backbone, and a DOTA chelator is synthesized to produce a compound with specificity for tumor-associated macrophages, capable of detection by single photon computed tomography (SPECT) or SPECT-CT and delivering local radiotherapy. The DOTA is attached to the backbone by a non-cleavable bond to allow for the accumulation of Lutetium-177 within the TAMs to deliver local radiation to the surrounding tumor cells. Lutetium-177 is chelated onto the DOTA ligand via the standard chelation method as detailed in Example 2 of U.S. Pat. No. 10,596,276 B2 and U.S. Pat. No. 10,596,278, the Example 2 of which are hereby incorporated by reference.

### Example 2. Radio Labelling of Compound A

Radiolabeling (RL) of Compound A is performed in a hotcell. Radiochemical purity is determined by instant thin layer chromatography silica gel and radio-high performance liquid chromatography. The determined radiochemical (RC) purity is expected to be >95%.

### Example 3. Radio Ligand Stability of Compound A

Compound A is incubated in saline or serum (37° C. timepoints up to 24 hours (hrs) followed by reversed-phase HPLC (RP-HPLC) to evaluate in vitro stability. The level of intact radioligand at 24 hr is expected to be >90%.

### Example 4. Biodistribution of Compound A

### SPECT or SPECT/CT Imaging

Compound A is prepared and injected into HT1080 tumor-bearing mice for imaging (n=24 overage, 12 mice in study, 3 groups of n=4/group of male and female). This is helpful for clinical translation as major organs and tumor are visualized.

### Gamma Counting

A second BD study is performed using ex vivo gamma counting of up to 10 tissues and tumor (n=18 overage for 9 mice in study, with 3 mice/time points). This methodology is more accurate and assesses smaller tissues than SPECT or SPECT/CT. After i.v. injection of Compound A, SPECT or SPECT/CT scans and gamma counting are performed, and uptake into organs as well as into tumor is calculated and monitored to determine retained activity as a percent of the injected activity at 4 hr, 24 hr, and 72 hr. Areas under the curve (AUCs) is determined based on non-decay corrected biodistribution data for selected organs and tissues. The ratio of the AUC values for the tumor relative to the organs respectively is calculated as an indirect measure for the absorbed dose ratios. The measured tumor to non-specific organ uptake of Compound A ratio is expected to be >5:1 by 24 hr.

### Example 5. Structural Stability of Compound A

NMR spectroscopy is used to determine the structural stability of Compound A. Seven samples of compound A were treated with DMSO under elevated temperatures of 35° C., 50° C., 60° C., 70° C., 80° C., 90° C., and 100° C., respectively, for 1 hour (see table below, Compound A #2 to #8). Characterization by NMR spectroscopy of these samples in DMSO show no extra-degradation peak in H NMR pattern as compared to the H NMR pattern of a sample of compound A in DMSO that was not treated under elevated temperature (see table below, Initial Sample, Room temp). Such results indicate compound A is stable under elevated temperatures.

| Input | Solvent | Temp (° C.) | Time (hr) | Comments |
|---|---|---|---|---|
| Compound A #1 | DMSO | Room temp | 0 | |
| (Initial Sample) | | | | |
| Compound A #2 | DMSO | 35 | 1 | H NMR pattern same as initial sample at RT. No |
| | | | | extra-degradation peak observed. |
| Compound A #3 | DMSO | 50 | 1 | H NMR pattern same as initial sample at RT. No |
| | | | | extra-degradation peak observed. |
| Compound A #4 | DMSO | 60 | 1 | H NMR pattern same as initial sample at RT. No |
| | | | | extra-degradation peak observed. |
| Compound A #5 | DMSO | 70 | 1 | H NMR pattern same as initial sample at RT. No |
| | | | | extra-degradation peak observed. |
| Compound A #6 | DMSO | 80 | 1 | H NMR pattern same as initial sample at RT. No |
| | | | | extra-degradation peak observed. |
| Compound A #7 | DMSO | 90 | 1 | H NMR pattern same as initial sample at RT. No |
| | | | | extra-degradation peak observed. |
| Compound A #8 | DMSO | 100 | 1 | H NMR pattern same as initial sample at RT. No |
| | | | | extra-degradation peak observed. |

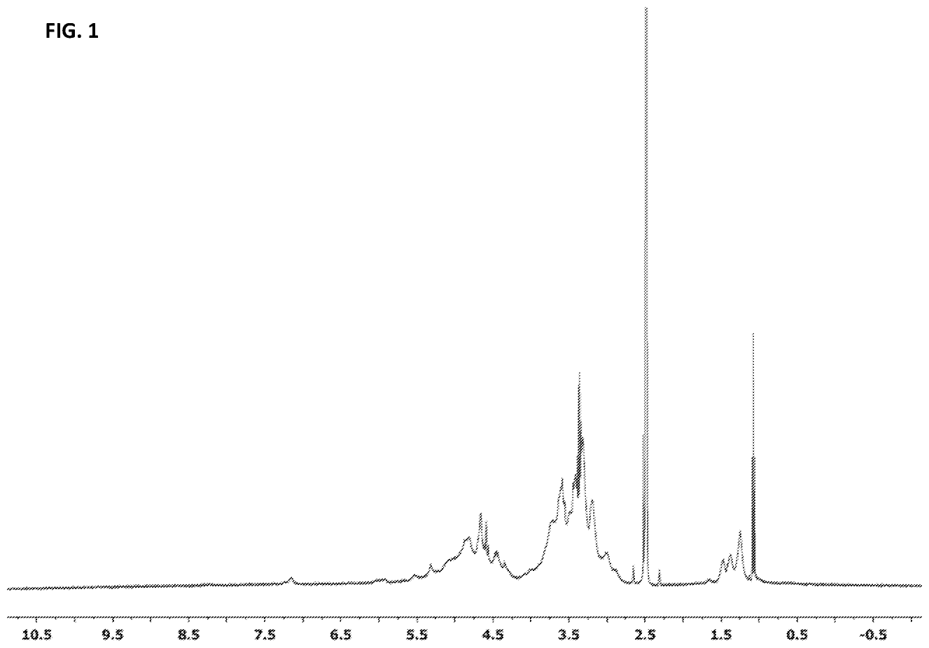

Stability is an important characteristic for effective chelation of radioactive isotopes. FIG. 1 shows the NMR spectra of Compound A.

1. A method of treating a solid tumor comprising administering to a subject in need thereof a composition, wherein the composition comprises:
i) a tumor-associated macrophage-targeting moiety;
ii) a glucan backbone, wherein the glucan backbone comprises a plurality of backbone monomers;
iii) an active component comprising a dodecane tetraacetic acid (DOTA) substrate, wherein the active component is coupled to the glucan backbone, and wherein the DOTA substrate is chelated to a radioisotope; and
iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

2. The method of claim 1, wherein the radioisotope is selected from the group consisting of: ⁶⁷Ga, ⁶⁸Ga, ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, ³²P, ²²⁵Ac, and ¹⁷⁷Lu.

3. The method of claim 1 or 2, wherein the radioisotope is ¹⁷⁷Lu.

4. A composition comprising:
i) a tumor-associated macrophage-targeting moiety;
ii) a glucan backbone, wherein the glucan backbone comprises a plurality of backbone monomers;
iii) an active component comprising a dodecane tetraacetic acid (DOTA) substrate, wherein the active component is coupled to the glucan backbone, and wherein the DOTA substrate is chelated to a radioisotope selected from the group consisting of: ²¹²Bi, ¹³¹I, ¹¹¹In, ⁹⁰Y, ¹⁸⁶Re, ²¹¹At, ¹²⁵I, ¹⁸⁸Re, ¹⁵³Sm, ²¹³Bi, and ³²P; and
iv) a targeting linker that links the tumor-associated macrophage-targeting moiety to the glucan backbone, wherein the targeting linker comprises a carbamate group and a chain moiety, wherein the carbamate group is connected to the backbone monomer and the chain moiety connects the carbamate group and the tumor-associated macrophage-targeting moiety.

5. The composition of claim 4 or the method of any one of claims 1 to 3, wherein the plurality of backbone monomers comprises a plurality of D-glucose monomers in a α-1,6 glycosidic linkage or a beta-1,4 glycosidic linkage.

6. The composition of claim 4 or 5 or the method of any one of claims 1 to 3, wherein the plurality of D-glucose monomers is n, wherein n=16 to 111.

7. The composition of any one of claims 4 to 6 or the method of any one of claims 1 to 3, wherein the plurality of D-glucose monomers is n, wherein n=50 to 65.

8. The composition of any one of claims 4 to 7 or the method of any one of claims 1 to 3, wherein the glucan backbone is a linear dextran molecule.

9. The composition of any one of claims 4 to 8 or the method of any one of claims 1 to 3, wherein the glucan backbone is a cyclodextrin molecule, wherein n=6 to 16.

10. The composition of any one of claims 4 to 9 or the method of any one of claims 1 to 3, wherein the tumor-associated macrophage-targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate.

11. The composition of any one of claims 4 to 10 or the method of any one of claim 1 to 3, wherein the tumor-associated macrophage-targeting moiety is a mannose.

12. The composition of any one of claims 4 to 11 or the method of any one of claim 1 to 3, wherein the ratio of mannose to backbone monomers is about 1 to 5 to about 1 to 25.

13. The composition of any one of claims 4 to 12 or the method of any one of claim 1 to 3, wherein the ratio of mannose to backbone monomers is about 1 to 6 to about 1 to 19.

14. The composition of any one of claims 4 to 10 or the method of any one of claim 1 to 3, wherein the degree of substitution of mannose on a cyclodextrin ranges from about 0.1 to about 7.

15. The composition of any one of claims 4 to 10 or the method of any one of claim 1 to 3, wherein the degree of substitution of mannose on a cyclodextrin ranges from about 0.5 to 5.

16. The composition of any one of claims 4 to 15 or the method of any one of claim 1 to 3, wherein the targeting linker is connected to the glucan backbone through the oxygen atom of the carbamate group.

17. The composition of any one of claims 4 to 16 or the method of any one of claim 1 to 3, wherein the chain moiety of the targeting linker comprises a C₃-C₇ alkylene chain.

18. The composition of any one of claims 4 to 17 or the method of any one of claim 1 to 3, wherein the chain moiety of the targeting linker comprises a C₆-alkylene moiety.

19. The composition of any one of claims 4 to 18 or the method of any one of claim 1 to 3, wherein the chain moiety of the targeting linker is a unsubstituted C₆-alkylene moiety.

20. The composition of any one of claims 4 to 19 or the method of any one of claim 1 to 3, wherein the carbon atom of the carbamate group of the targeting linker is the only sp2-hybridized carbon when said linker is attached to mannose.

21. The composition of any one of claims 4 to 20 or the method of any one of claim 1 to 3, wherein the active component is coupled to the glucan backbone via a payload linker.

22. The composition or method of claim 21, wherein the payload linker is a non-cleavable linker.

23. The composition of any one of claims 4 to 22 or the method of any one of claims 1 to 3, wherein the tumor-associated macrophage-targeting moiety is a moiety targeting CD205 (DEC205), CD206, CD207 (langerin), CD209 (DC-SIGN), CD280 (ENDO180), or CD301 (MGL).

24. The composition of any one of claims 4 to 23 or the method of any one of claims 1 to 3, wherein the composition has a molar ratio between the tumor-associated macrophage-targeting moiety and the active component from about 1:1 to about 1:10 or from about 1:1 to about 1:6.

25. A method of treating a tumor or a granulomatous disease in a subject in need thereof, comprising administering to the subject a therapeutically effective amount of the composition of any one of claims 4 to 24.

26. The method of any one of claim 25, wherein the tumor is a benign tumor.

27. The method of claim 26, wherein the benign tumor is a grade 1 meningioma, grade 2 meningioma, or grade 3 meningioma.

28. The method of claim 25, wherein the granulomatous disease is tuberculosis.

29. The method of any one of claims 1 to 3 or 25, wherein the tumor is sarcoma, lymphoma, carcinoma, blastoma, germ cell tumor, B cell lymphoma, T cell lymphoma, mycosis fungoides, Hodgkin's Disease, myeloid leukemia, multiple myeloma, bladder cancer, brain cancer, nervous system cancer, head and neck cancer, squamous cell carcinoma of head and neck, kidney cancer, lung cancers, small cell lung cancer, non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, triple negative breast cancer, epithelial cancer, renal cancer, genitourinary cancer, pulmonary cancer, esophageal carcinoma, head and neck carcinoma, large bowel cancer, hematopoietic cancers testicular cancer; colon and rectal cancers, prostatic cancer, gliosarcoma, Kaposi sarcoma, esophageal cancer, hepatocellular cancer, or pancreatic cancer.

30. The method of claim 29, wherein the tumor is sarcoma or glioblastoma.

31. The method of claim 29 or 30, wherein the tumor is soft tissue sarcomas.

32. A method of treating a solid tumor in a subject in need thereof, comprising administering to the subject a therapeutically effective amount of the composition of any one of claims 4 to 24.
